(19) United States
(12) Patent Application Publication
(10) Pub. No.: US 2014/0031282 A1
(43) Pub. Date: Jan. 30, 2014

(54) FORMULATION FOR TREATMENT OF HYPERINSULINEMIC HYPOGLYCEMIA FOLLOWING GASTRIC BYPASS SURGERY

(71) Applicant: Emily L. Cooper, Shoreline, WA (US)

(72) Inventor: Emily L. Cooper, Shoreline, WA (US)

(21) Appl. No.: 13/937,010

(22) Filed: Jul. 8, 2013

Related U.S. Application Data
(60) Provisional application No. 61/668,797, filed on Jul. 6, 2012.

(51) Int. Cl. A61K 31/702; A61K 38/26

(52) U.S. Cl. CPC A61K 31/702; A61K 38/26
USPC 514/7.2

(57) ABSTRACT

An effective combination of 6.25-100 mg of acarbose with a GLP-1 mimetic, such as 5-10 mg exenatide or 0.3-3.0 mg liraglutide.

### TECHNICAL FIELD

This invention relates generally to the treatment of certain negative consequences of gastric bypass surgery, and more particularly concerns the treatment of hyperinsulinemic hypoglycemia occurring following gastric bypass surgery.

### BACKGROUND OF THE INVENTION

It is well known that obesity, both in adults and children, is increasing in the United States and is becoming a substantial concern to medical professionals. In some extreme cases various types of surgery are used to reduce and control weight. The number of such surgeries has increased significantly over the past few years, to the point where approximately 200,000 surgeries are performed each year, with the number expected to continue to increase.

Gastric bypass surgery, however, is not without its complications, risks and negative consequences. One such complication, while relatively rare (reportedly occurring in approximately 1% of surgeries), is severe, although incidence is believed to be 2-7 times that number when patients who have either not undergone surgery or had other types of bariatric surgery are included, and has quite a negative effect on the quality of life and well-being of the patient. This condition is known as hyperinsulinemic hypoglycemia (which can be associated with nesidioblastosis). Other causes can lead to hyperinsulinemic hypoglycedmia as well. This generally refers to after meal spikes in insulin with resulting extreme drops in blood sugar, where the patient experiences significant negative effects, including extreme sleepiness and fatigue, anxiety, in some cases a confusional state and passing out, or even seizures in extreme cases.

Although there is often a family history of Diabetes Type 2 or a pre-diabetes condition in such patients, it is not always the case. In treatment of this condition, alpha glucosidase inhibitors, such as acarbose, have been tried, but with only limited success. In other cases, surgical pancreatic resection has been recommended. In many other cases, however, an accurate diagnosis is missed, with the symptoms being interpreted as panic disorder or depression; attempts at treatment typically include the use of medications suitable for those conditions. Such attempts have not been very successful. Hence, there is a significant need for an effective treatment of patients experiencing the particular condition of post-prandial hyperinsulinemic hypoglycemia following gastric bypass surgery.

### BEST MODE FOR CARRYING OUT THE INVENTION

The treatment disclosed herein is directed toward post Roux-en-Y bariatric surgery patients, but other patients as well, such as those who may not have had the surgery, who experience post-prandial hyperinsulinemic hypoglycemia, specifically spikes in insulin following meals with resulting severe drop in blood sugar. The typical recommendation is to eat smaller meals more frequently, but since the lowest glucose levels occur between 30-90 minutes after eating, it is quite difficult to eat frequently enough to prevent the resulting severe dip in glucose responsible for the condition.

The present treatment does not involve an eating plan or other behavior modifications. The treatment comprises a specific combination of acarbose with a GLP-1 (glucagon-like peptide-1) mimetic. Acarbose has been used for the treatment of Type 2 Diabetes and is marketed as Precose, in tablet form. While acarbose has been used alone in the treatment of the above condition, as indicated above, it has not been particularly successful. I have determined that an effective treatment is a combination of a selected effective amount of acarbose with a selected effective amount of a GLP-1 mimetic. Examples of an effective GLP-1 mimetic are exenatide, sold by Amylin Pharmaceuticals, or liraglutide, sold by Novo Nordisk, currently sold in injectable form.

In the present case, 6.25-100 mg of acarbose is combined with 5-10 mg of exenatide or 0.3-3.0 mg liraglutide. A most preferred combination is 25 mg of acarbose with 5 mg of exenatide. Other known GLP-1 mimetics can be used or others as they are developed. Other alpha glucosidase inhibitors in place of acarbose could also be used. Further, other medications or supplements, such as DPP-4 inhibitors (also called gliptins), which prolong the effect of GLP-1 and GIP, or Ptamintide, which is an amylin mimetic which can delay gastric emptying, may be added to the formulation to enhance the effectiveness or usefulness of the formulation. Each mimetic will have its own specific dose in combination with a selected dose of acarbose. Dosing is set according to the recommendation of the drug manufacturer—exenatide is 10-60 minutes prior to a meal, liraglutide usually in the morning prior to breakfast, and acarbose with the first bite or immediately preceding the first bite of every meal, for example.

I have found that the above formulation results in a normal glucose and insulin response post-prandially as opposed to the results of same meal being consumed without the formulation.

Accordingly, an effective formulation has been discovered for the treatment of post-prandial hyperinsulinemic hypoglycemia.

Although a preferred embodiment of the invention has been disclosed for purposes of illustration, it should be understood that various changes, modifications and substitutions may be incorporated in the embodiment without departing from the spirit of the invention, which is defined by the claims which follow.

What is claimed is:

1. A formulation for treatment of post-prandial hyperinsulinemic hypoglycemia comprising an effective amount of acarbose or other alpha glucosidase inhibitor in combination with an effective amount of a GLP-1 mimetic, resulting in a normal glucose and insulin response post-prandially.
